(19) United States
(12) Patent Application Publication
(10) Pub. No.: US 2017/0112452 A1
(43) Pub. Date: Apr. 27, 2017

(54) Systems, Devices, and/or Methods for Identifying Risk of Severe Hypoglycemia in the Next 24 Hours

(71) Applicant: Erik A. Otto, Charlottesville, VA (US)

(72) Inventor: Erik A. Otto, Charlottesville, VA (US)

(21) Appl. No.: 15/331,829

(22) Filed: Oct. 22, 2016

Related U.S. Application Data
(60) Provisional application No. 62/244,899, filed on Oct. 22, 2015.

(51) Int. Cl. A61B 5/00; G06F 19/00; A61B 5/145

(52) U.S. Cl. CPC A61B 5/7275; A61B 5/14532; A61B 5/486; A61B 5/7221; A61B 5/746; A61B 5/7475; G06F 19/3431; G06F 19/322; G06F 19/3468; G06F 19/3475

(57) ABSTRACT

Certain exemplary embodiments can provide a method, which can comprise, via an input/output device, rendering an alert of hypoglycemia risk. The hypoglycemia risk can be determined based upon a strength of an automatically determined pattern in blood glucose data. The blood glucose data can comprise new blood glucose data and/or a set of pre-existing blood glucose data. The automatically determined pattern can be determined based upon a calculation of at least two metrics.

### CROSS-REFERENCES TO RELATED APPLICATIONS

This application claims priority to, and incorporates by reference herein in its entirety) pending U.S. Provisional Patent Application Ser. No. 62/244,899 (Attorney Docket No. 1049-005), filed Oct. 22, 2016.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 is a diagram of exemplary hardware and functional components of a system;

FIG. 2 is a flowchart of an exemplary embodiment of a method;

FIG. 3 is a chart of an exemplary embodiment of a method of retrospective presentation of information produced from the method;

FIG. 4 is an exemplary embodiment of the presentation of an output of the method;

FIG. 5 is a block diagram of an exemplary embodiment of an information device.

### DETAILED DESCRIPTION

Certain exemplary embodiments can provide a method, which can comprise, via an input/output device, rendering an alert of hypoglycemia risk. The hypoglycemia risk can be determined based upon a strength of an automatically determined pattern in blood glucose data. The blood glucose data can comprise new blood glucose data and/or a set of pre-existing blood glucose data. The automatically determined pattern can be determined based upon a calculation of at least two metrics.

A basic problem that people with diabetes have relates to the transfer of sugar, contained in the blood, across cell membranes. This in turn makes it difficult for the body to maintain sugar levels in the blood at the correct level. In the treatment of diabetes, patients regularly check blood glucose levels using a self-testing kit. By comparing the result of a self-test with the blood glucose level considered normal, a patient is able to estimate the amount of insulin that should be taken to keep the blood glucose level near normal, or what to eat to bring blood glucose into equilibrium, or how much to exercise. Too much blood sugar (e.g. due to the patient injecting too little insulin or eating more than the prescribed amount) is defined as hyperglycemic, while too little blood sugar (e.g. due to the patient injecting too much insulin) is defined as hypoglycemic. These are considered to be short-term complications of diabetes and can cause acute symptoms or be a factor in the development of long-term complications. Diabetic patients can also suffer problems arising from their condition that only become apparent in the longer term. These problems include retinopathy, peripheral neuropathy, nephropathy and an increased risk of a number of cardiovascular events comprising stroke and myocardial infarction.

In order to monitor blood sugar levels, people with diabetes can perform self-monitoring of blood glucose (“SMBG”) periodically to obtain information about their blood glucose levels using a handheld or implanted blood glucose monitoring device. This information is used by patients and clinicians to guide treatment and management decisions so that blood glucose levels can be kept in an optimal range to avoid hypoglycemia and hyperglycemia.

Testing is performed several times a day by most patients on insulin, but even with frequent testing there can be a lack of information as glucose levels fluctuate significantly throughout the day. Moreover, patients might not have readily available information or analytical tools providing insight about what might be the best time of day to test, or what time of day there might be problematic patterns that warrant a greater frequency of monitoring or increased diabetes management attention.

Patients may achieve better outcomes by obtaining information on their condition in real time, e.g., when they are making management decisions, so that the information is immediately actionable, without the need for patients to analyze detailed charts and graphs retrospectively.

Continuous glucose monitoring (“CGM”) systems can provide frequent (e.g., approximately every 1-20 minutes) feedback on blood glucose control levels in real-time, such as via an imbedded cannula and a portable device with display (in addition to a SMBG meter). However, CGM devices can be more expensive than conventional SMBG meters, and can utilize additional device implantation or calibration.

Hypoglycemia is a serious medical condition caused by abnormally low blood glucose (“BG”), with symptoms ranging from mild (e.g. shakiness, sweating, heart pounding) to severe, (e.g. seizures, unconsciousness, and even death). Consequently, hypoglycemia has been determined as a major barrier to improving glycated hemoglobin (“HbA1c”) and reducing the risks of long-term complications. Therefore predictive methods are desired that can identify patterns of blood glucose that tend to precede severe hypoglycemia, and that effectively warn patients of increased risk before an episode occurs, preferably in real-time or contemporaneous with the period of risk.

We present a novel method for identifying imminent risk of hypoglycemia. This method incorporates elements of predictive transformations that have been previously disclosed, but also incorporates novel elements and combinations of elements. Kovatchev, Cox et al. have previously disclosed transformations of blood glucose data, wherein two logarithmic clinical risk symmetrization functions are applied to blood glucose data below approximately 112.5 mg/dL, on the one hand, and above 112.5 mg/dL on the other. An evaluation of the extent and frequency of lows named the Low Blood Glucose Index (“LBGI”) can be performed using a derivation of symmetrized blood glucose readings below approximately 112.5 mg/dL.

Another metric called the Average Daily Risk Range (“ADRR”) is a derivation of symmetrized blood glucose readings that is indicative of overall blood glucose variability on both ends of the blood glucose scale. LBGI and, to some extent, ADRR can be employed to predict the risk of hypoglycemia, however more sophisticated heuristic and Boolean methods incorporating multiple LBGI and ADRR thresholds combinations have not been determined, nor have Boolean combinations with other methods such as a simple percent of readings considered hypoglycemic within a given time period. In our exploratory evaluation of various methodologies, a unique Boolean combination of these elements revealed improved predictive performance, and thus prompted us to develop it as a hypoglycemic-predictive method.

Our hypothesis for using multiple thresholds and Boolean combinations of methodologies is that we may capture more at-risk situations by including patterns forming with extremes in either ADRR or LBGI when the other measure may be more stable. These additional at-risk periods may not otherwise be captured by using a static threshold for LBGI and ADRR individually or in combination. We also have included a Boolean combination of a minimum percent of readings in the hypoglycemic range (≦approximately 70 mg/dL) with each set of ADRR and LBGI thresholds, based on supplementary retrospective analyses of complementary methodologies in a large dataset.

Certain exemplary embodiments of this invention provide a method, system, and/or computer program product related to the maintenance of improved control of diabetes, directed to determining patterns indicative of a risk of near term (within the next 24-48 hours) of hypoglycemia or severe hypoglycemia. A user can be notified of the pattern indicative of risk of hypoglycemia in a time period precedent to or during the period of risk, so that a detrimental pattern can be avoided or mitigated by the user through hypoglycemia avoidance diabetes management actions such as increasing testing frequency, adjusting medication dose, adjusting food intake, adjusting activity levels, enlisting support from others, checking more frequently for hypoglycemia symptoms, and/or being prepared to administer a glucagon injection. The method, system, and/or computer program product pertain directly to the enhancement of diabetes management software and hardware, including software that resides on devices such as blood glucose measurement devices, wearable medical or wellness devices, other device systems, and/or networked computer systems.

Certain exemplary embodiments provide real-time information to the user about upcoming periods of hypoglycemic risk. This real-time information is directed to providing insight to the user such that the user can take appropriate action, such as increased testing in a designated time period, thereby leading to better glycemic control. Either during daily times that are preprogrammed, or during the blood glucose measurement process and prior to the presentation of a blood glucose result, certain exemplary embodiments evaluate historical patterns of glycemia and, based on this evaluation, issues warnings about patterns indicative of risk of hypoglycemia either immediately in real-time, or at the time a glucose measurement is presented, or in a future time period when the period of risk has been identified.

Certain exemplary embodiments may tabulate notifications of patterns of hypoglycemic risk and present these tabulations retrospectively back to the user. These tabulations may present the number of notifications of patterns indicative of risk over various time periods, such as over several days or weeks. Tabulations of notifications may also include supplementary information relating to the blood glucose readings preceding and following notifications, or diabetes management actions taken by the user leading up to, during and following the notification or period of risk. Tabulated user actions may include various diabetes managements such as increasing or decreasing insulin, eating more or less carbohydrates, or altering activity levels. Actions may also include the adoption of more frequent monitoring.

Certain exemplary embodiments may notify a caregiver or healthcare organization about imminent risk of hypoglycemia for a patient when the period of risk has been identified. This message could be rendered via electronic communication such as email, text messaging or other notifications. The alert could also be rendered in a summary display or device interface that includes information about multiple patients and their glycemic status. On the summary screen additional risk messages may be conveyed about one or more patients, such as risk of severe hyperglycemia, risk of other acute risks, or information about comorbidities or medication adherence.

Certain exemplary embodiments may also present a timeline of several hours or days of continuous periods of persistent hypoglycemia risk, wherein one or more notifications about hypoglycemia have been provided. This timeline may include management or blood glucose information, including highlighting of any readings that may be considered hypoglycemic, or of recorded symptomatic hypoglycemic events.

Certain embodiments may suggest and/or require sufficient SMBG or CGM testing over specific predefined periods of time for the hypoglycemia risk warning to be effective. Suggestions and/or requirements can range from an average of approximately one time per day over a predefined period to over, for example, 10,000 times per day for CGM data streams.

Other components of the Boolean methods for identifying patterns indicative of hypoglycemia can also, or alternatively, comprise other forms that are indicative of a high frequency of hypoglycemia within a time period, or relative to a general population. For example, methods could include identifying a count of two or more blood glucose readings that are below a given clinical threshold in the preceding days, or evaluating if the average blood glucose level in that time period over a predetermined time are below a predefined threshold.

The method of the invention may be implemented using hardware, such as hardware specific processors and/or integrated circuits, and software such as computer programs and/or computer program logic, and devices with communication infrastructure and/or blood glucose measurement systems or a combination thereof and may be implemented in one or more computer systems or other processing systems, such as a smartphone, wearable device, medical device, and/or a blood glucose monitoring device equipped with adequate processing capabilities and memory.

In certain exemplary embodiments, a user can receive feedback on patterns indicative of risk of severe hypoglycemia on their blood glucose monitoring system or a data management system. A data management system in accordance with the present invention can comprise a personal digital assistant (“PDA”), phone, smartphone, iPhone®, blood glucose monitoring device, tablet, watch, insulin pump, insulin dosing device, internet interface, optical viewing device, wearable device, medical device, device system, an autonomous agent of the user, and/or other media, etc. The evaluation of patterns can also be performed remotely from the user's device system or data management system, and then the feedback given at a time when the user has access to their device or data management system.

Certain embodiments will have a variety of user-selectable settings for the frequency of alerts for hypoglycemic risk. Settings generating less frequent alerts and notifications can have a greater specificity and thus greater predictive power, and conversely more frequent messaging settings can result in greater sensitivity but lesser predictive power. Prior to engagement of any blood glucose testing action, risk calculation or risk alerting, the user will preferably have had the option of selecting from a one to several frequency settings, preferably two to five, or to select from a range of frequency scale.

The system will identify hypoglycemia risk periods and notify the user using a series of predefined steps. These steps do not necessary need to be followed sequentially, nor to all steps need to be performed. Embodiments as described below comprise enumerated steps.

Step 1: In a preferred embodiment, the patient tests their blood sugar using a SMBG or CGM device. This process can involve the sampling of blood and/or interstitial fluid of the patient, and then measuring glucose concentration using electro-chemical, infrared, photoelectric, or other means. New blood glucose information can be added to a database of pre-existing blood glucose data. If the database is not accessible, the system can retrieve blood glucose data to have a window of data for evaluating risk of hypoglycemia. This window of retrieved data can be as little as approximately twenty-four hours or as much as a year or more, but is preferably approximately two to four weeks of data. In a preferred embodiment, the CGM or SMBG data can be collected via real-time wireless communication with a medical device system or glucose monitoring device.

The acceptance of new blood glucose data triggers an evaluation of the risk of near term hypoglycemia in the next 24-48 hours. The method can be enacted using the processor of a monitoring device, an ancillary wearable device, a portable device, a telephonic device or a computer with a non-transitory media. The evaluation of the risk of near term hypoglycemia may also be triggered at pre-programmed times, or at defined intervals, in the event new blood glucose data is not received at those times.

Step 2: The system evaluates the total number of glucose readings in a prior time period (the “evaluation window”) to determine if there is sufficient data to evaluate a period of hypoglycemia risk. This evaluation window could range from approximately two (2) days to approximately (60) days, preferably approximately the last fourteen (14) days (starting from the current reading, comprising any previous readings on the current day, and going back approximately the last 14 calendar days). Preferably there should be 35 or more BG Readings taken in the cumulative period including the current day and the previous 14 Days. The system may desist from evaluating hypoglycemia risk if there is a deficiency of data in the evaluation time period for example, if there are less than approximately (5) readings in previous 48 hours (this time window is taken from the time of evaluation and going back, for example, approximately 48 hours). In the event there is a deficiency of data noted, the system may issue a visual or auditory message about the lack of sufficient data for evaluation of one or more periods of hypoglycemia risk to the user on a diabetes management or wellness device such as a wearable, insulin pump, glucose monitor, telephonic system, tablet computer, laptop, desktop computer, and/or other device system.

Step 3: The system can calculate risk values within a specified time window similar to or exactly the same as the predefined evaluation window of approximately the prior two (2) to sixty (60) days. In an exemplary embodiment: N₁₄=the number of readings in the previous 14 Days, including any readings on the current day.

For all BG readings from i to N₁₄ in the previous 14 Days, including any readings on the current day, perform the following calculations:

scaleᵢ=1.509*((ln(BGᵢ))^{1.08405}−5.381))

Note: If natural logarithms are difficult to calculate, a look up table can be used for glucose values (plasma calibrated) from 20 to 600 (each one an integer)

riskᵢ=10(scaleᵢ)²

If BGᵢ<=112.5 mg/dL, then RiskLoᵢ=riskᵢ, else RiskLoᵢ=0
If BGᵢ>112.5 mg/dL, then RiskHiᵢ=riskᵢ, else RiskHiᵢ=0

Step 4: The system can calculate one or more measures of the degree and frequency of low blood glucose readings within another evaluation window in the last several hours to 60 days. In an exemplary embodiment a two (2) day LBGI is calculated:

N_{24H}=the number of readings in approximately the last 24 hours (right up until the time of evaluation). For example, if the LBGI calculation is being done at approximately 2:05 pm, this should be the number of readings in the period from approximately 2:05 pm on the previous day until approximately 2:05 pm on the current day. This period of time is called PreHRI_{24H}
N_{DBPD}=a count of readings in the 24 hour period immediately preceding the last 24 hours. (The 24 hours before PreHRI_{24H})

LBGI_{2D}=(1/(N_{24H}+N_{DBPD}))*[(the sum of all RiskLo values for all readings in the the PreHRI_{24H} period)+(the sum of RiskLo values for all readings in the 24 hour period immediately preceding the PreHRI_{24H} period)]

Step 5: The system can calculate one or more patient metrics indicative of the degree of glucose oscillation or glucose variability across high and low ends of the glucose scale. The overall variability of a person can be computed using the ADRR (average daily risk range), but can be also computed using the overall standard deviation of blood glucose readings, M-value (as defined in Schlichtkrull J, Munck O, Jersild M “The M-value, an index of blood-sugar control in diabetics”, Acta Med Scand 177:95-102, 1965), mean amplitude of glycemic excursions (“MAGE”), Lability Index (as defined in Edmond A. Ryan, Tami Shandro, Kristy Green, Breay W. Paty, Peter A. Senior, David Bigam, A. M. James Shapiro, and Marie-Christine Vantyghem, Assessment of the Severity of Hypoglycemia and Glycemic Lability in Type 1 Diabetic Subjects Undergoing Islet Transplantation, Diabetes April 2004 vol. 53 no. 4 955-962), and/or any other measure of variability. The standard deviation of blood glucose readings can make the variability profile more sensitive to hyperglycemic excursions and less sensitive to hypoglycemia. In an exemplary embodiment, we used the ADRR because this measure of variability can be superior in terms of its sensitivity to, and predictive ability of extreme glycemic excursions, and because it has a clearly identified population threshold.

In an exemplary embodiment, for each day in the 14 day period calculated back from the time of the current reading (all readings on the current day, plus approximately the previous 14 calendar days), calculate a daily risk range; M=1 to 15=the number of days for calculation of the Daily Risk Range DailyRiskRange_{M}=For each day in approximately the last 14 days (including the current day), the maximum RiskLo value plus the maximum RiskHi value.

DailyRiskRange_{M}=max RiskLo (all readings on day M)+max RiskHi (all readings on day M)

Average Daily Risk Range (“ADRR_{HRI}”)=For every day that had BG Readings (number of qualifying days=N), take the sum of all DailyRiskRange over approximately the last 14 days (and the current day) and divide the total by N.

Step 5: The system can calculate one or more patient metrics indicative of the proportion or relative distribution of glucose readings that are considered hypoglycemic within a given evaluation window. This evaluation window may differ between various calculation methodologies.

In an exemplary embodiment, for each reading calculated back from the time of the current reading (all readings on the current day, plus approximately the previous 14 calendar days), calculate the percentage of readings that are less than 70 mg/dL;

N₁₄=Total number of readings in the current Day and over approximately the last 14 calendar Days prior to the current Day.
CountLows₁₄=Total number of readings that are <70 mg/dL in the current Day and over approximately the last 14 calendar Days prior to the current Day.

PERCLOW₁₄=CountLows₁₄/N₁₄

Step 6: The system can access predefined thresholds for providing a flag based on the user settings.

These thresholds may change depending on user preference for the frequency of messaging, and can be pre-set prior to Step 1.

LBGI_{Thresh1}=4, or preferably between approximately 3 and approximately 5
ADRR_{Thresh1}=30, or preferably between approximately 20 and approximately 40
LBGI_{Thresh2}=3, or preferably between approximately 2 and approximately 4
ADRR_{Thresh2}=40, or preferably between approximately 30 and approximately 50
LBGI_{Thresh3}=5, or preferably between approximately 4 and approximately 6
ADRR_{Thresh3}=26, or preferably between approximately 20 and approximately 32
PERCLOW_{Thresh1}=0.19, or preferably between approximately 0.15 and approximately 0.24
PERCLOW_{Thresh2}=0.24, or preferably between approximately 0.19 and approximately 0.29

These thresholds may vary depending on the desired sensitivity, specificity and messaging frequency of the alert for a particular user, as well as the nature of the patients diabetes control, type of diabetes. The above preferred threshold settings have been identified through analysis of large amounts of patient data, literature reviews, and through examination of accepted clinical practice guidelines.

Step 7: The system can determine if the assigned thresholds are exceeded and/or a message should be issued. In a preferred embodiment, a pattern indicative of a period of risk for severe hypoglycemia can be identified IF;

LBGI_{2D} is greater than or equal to LBGI_{Thresh1} AND ADRR_{HRI} is greater than or equal to ADRR_{Thresh1} AND PERCLOW₁₄ is greater than or equal to PERCLOW_{Thresh1}

### OR

if LBGI_{2D} is greater than or equal to LBGI_{Thresh2} AND ADRR_{HRI} is greater than or equal to ADRR_{Thresh2} AND PERCLOW₁₄ is greater than or equal to PERCLOW_{Thresh1}

### OR

if LBGI_{2D} is greater than or equal to LBGI_{Thresh3} AND ADRR_{HRI} is greater than or equal to ADRR_{Thresh3} AND PERCLOW₁₄ is greater than or equal to PERCLOW_{Thresh1}

### OR

if LBGI_{2D} is greater than or equal to LBGI_{Thresh2} AND ADRR_{HRI} is greater than or equal to ADRR_{Thresh3} AND PERCLOW₁₄ is greater than or equal to PERCLOW_{Thresh2}

If a pattern of risk for hypoglycemia is identified, a message can be issued to the user. Otherwise, if no pattern is identified, it might be that no message is issued to the user, or a message might be issued to the user indicating that no pattern has been identified.

Step 8: Messaging. If a pattern of hypoglycemia risk has been identified, a message about hypoglycemia risk in the predefined time period can be issued to the user, or saved for future delivery to the user at a time during or preceding the period of risk. The message can also be delivered with information about methods of mitigating hypoglycemic risk, such as testing more frequently, applying continuous glucose monitoring, carrying fast-acting sugars, carrying a glucagon injection kit, avoiding intense exercise, and/or modifying the user's insulin regimen.

To evaluate the utility of the method individually and relative to other methods of identifying periods of risk for hypoglycemia, we conducted an in-silico study. The premise of this study is that any pattern that identifies periods of risk of severe hypoglycemia should be more predictive of future severe hypoglycemia relative to periods when no pattern has been identified. In addition, the prediction should be done sufficiently frequently to capture as many severe lows as possible, without warning the patient so frequently that the warnings become obtrusive.

In this study we evaluated the ability for our novel algorithm, and several alternative methods, to accurately flag for imminent risk of severe hypoglycemia. Performance assessments were based on prediction of Biochemical Severe Hypoglycemia (BSH is defined as BG≦approximately 40 mg/dL recorded on the patient's meter) and hypoglycemia (<approximately 70 mg/dL recorded on the patient's meter) within approximately the next 24 hours. This should be distinguished from severe hypoglycemia defined as “An event requiring assistance of another person to actively administer carbohydrate, glucagon or other resuscitative actions.” We chose BSH because 1) there is a paucity of available data for severe hypoglycemia not identified by a meter, 2) deep lows below approximately 40 mg/dL should be avoided in any case, and 3) we believe BG≦approximately 40 mg/dL should be low enough to possibly indicate compromised counter-regulatory function or Hypoglycemia Associated Autonomic Failure.

In our study we chose a prediction window of approximately 24 hours because we knew from prior studies that risk of a severe low could follow within a timescale of hours due to depleted counter-regulatory ability. In addition it is a timeframe in which people can actively take steps to modify behavior constructively for a possible intervention such as testing more frequently, bringing more fast-acting carbohydrates with them, checking for symptoms of hypoglycemia more often, or initiating CGM. Lastly, we hypothesized that, in the absence of further hypoglycemia after the flag, approximately 24 hours would give a sufficient window for the body to begin replenishing counter-regulatory hormones, hepatic glycogen, or awareness of hypoglycemia symptoms after any recent transient lows that may have contributed to triggering a warning of increased risk of severe hypoglycemia.

Development of the method was performed using a dataset of 297 patients with 240,241 SMBG readings (the “Test dataset”), the parameters frozen, and then it was applied to a dataset of 297 patients with 242,523 readings (the “Validation dataset”). More detail on these datasets can be found in Table 1 below. The resulting method will hereinafter be named Method 1.

TABLE 1
Demographics of the patients in the Test and Validation datasets.

| | | Validation |
|---|---|---|
| Demographic | Test Dataset | Dataset |
| Type Of Diabetes (Type 1/Type 2) | 130/161 | 129/162 |
| Test Frequency (Mean ± SD) | 3.84 ± 1.55 | 3.92 ± 1.62 |
| Number of BG | 240341 | 242523 |
| Number of BG ≦70 mg/dL | 20142 | 19345 |
| Number of BG ≦40 mg/dL | 1544 | 1433 |
| Baseline HbA1c (Mean ± SD) | 8.21% ± 1.38% | 8.17% ± 1.40% |
| Age (≦18/≧19 and ≦50/≧51)* | 40/55/32 | 27/62/46 |
| Gender (Male/Female) † | 86/76 | 88/75 |

The data in the Test and Validation datasets have been amalgamated from a data collection study, blood glucose monitoring device evaluations and a number of other behavioral evaluations occurring from 1998 to 2005, and then randomized into the Test and Validation datasets. All patients in the datasets were on either multiple daily injections or insulin pump therapy. “*” Indicates that age data was only available for 127 patients in the Test dataset and 135 patients in the Validation dataset. “†” Indicates that gender data was only available for 162 patients in the Test dataset and 163 patients in the Validation dataset.

Development of Method 1 involved evaluating flagging frequency, percent of BSH predicted in the 24-hour periods after a flag, and relative probability of at least one BSH episode occurring after a flag is raised for a number of different algorithm models and parameters. Alternative flagging criteria that were tested included counts of hypoglycemia, percentage of hypoglycemia, LBGI values and variability indices, sometimes in combination, across a number of time windows. Receiver Operating Characteristic across the breadth of flagging frequencies for these models was also evaluated. The analysis led to design enhancements to the base algorithm and selection of optimal models to be used for comparative purposes.

After testing and optimization in the Test dataset, the final Method 1 parameters were evaluated in the Validation dataset across all patients as well as cohorts of Type 1 and Type 2 patients, and glycemic control groups (Group 1=HbA1c<7%, Group 2=HbA1c≧7% and <9%, Group 3=HbA1c≧9%) at several flagging frequency rates (i.e. sensitivity settings). Multiple flagging frequency rates were evaluated because some work has shown some poor user acceptance at high flagging rates and thus we wanted patients to have the option of personalizing flagging rates to a lower frequency level. We also evaluated performance of Method 1 at various test frequency requirement ranges to characterize the impact of test frequency on performance.

Comparative pattern identification methods included Method 2: a simple standard deviation of readings over approximately the last 14 days, Method 3: a standard deviation and predetermined percent of readings that are hypoglycemic over approximately the last 14 days, Method 4: separate 14 day and 2 day LBGI calculation thresholds similar to a prior published algorithm, and Method 5: 14 day ADRR and 2 day LBGI calculation thresholds similar to another prior published algorithm.

Comparative Methods 2 and 3 were chosen because standard deviation is sometimes referred to as the most common tool for measuring blood glucose variability, and glycemic variability is often associated with hypoglycemia risk. In addition, evaluation of hypoglycemia risk may take the form of a percent methodology in clinical practice. Methods 4 and 5 represent advanced published methods incorporating LBGI and ADRR. Note that a 14-day BG window was used for comparative count, percent, ADRR and variability methods because in the development phase this window was found to be more optimal than windows of approximately seven (7) day or approximately thirty (30) day.

To evaluate Method 1 versus alternative methods we determined the relative probability and the amount of BSH predicted for all methods at the threshold trigger levels that would result in the closest possible flagging frequency to Method 1's default flagging frequency settings.

Given that Method 1 and the comparative methods could have many different values of percentages, variability indices, and/or LBGI to trigger a flag, methods were evaluated holistically across a range of trigger thresholds and warning frequencies. To do this we used a method previously described in our evaluation of a diurnal pattern messaging system comprising determining the partial area under the receiver operating characteristic curve (“AUROC”). The partial AUROC integrated the composite sensitivities and specificities of each method across a range of useful messaging frequencies so they could be compared as an area under the curve.

We chose to evaluate AUROC for specificities of 0.875 or greater. This threshold was based on the previously referenced evaluation of a hypoglycemia risk indicator algorithm in a clinical study where the algorithm in the study had an in-silico messaging frequency that delivered a specificity of approximately 0.875. Given that user feedback demonstrated some lack of user acceptance due to obtrusiveness at this level of specificity, we believe this presented an acceptable minimum level because the range of specificities evaluated will generally encompass a range of lesser flagging frequencies and have a lower number of false positives.

The performance for Method 1 across our desired sensitivity settings is shown in Table 2 below.

TABLE 2
Method 1 Performance across four flagging frequency settings,
presented in the order highest to lowest flagging frequency.

| | | | |
|---|---|---|---|
| | | Relative | |
| | Percentage | Probability of a | |
| Evaluable | of Days | BSH within 24 | Percentage of |
| Patients | with Flag | Hours of Flag | BSH Predicted |

HIGH (MOST SENSITIVE SETTING)

| | | | | |
|---|---|---|---|---|
| ALL DATA | 297 | 9.6% | 5.57 | 53.5% |
| TYPE 1 | 129† | 14.8% | 3.36 | 49.6% |
| TYPE 2 | 162† | 7.8% | 7.16 | 56.1% |
| BG GROUP 1 | 60 | 15.7% | 4.70 | 73.6% |
| BG GROUP 2 | 160 | 10.4% | 3.76 | 39.0% |
| BG GROUP 3 | 77 | 4.3% | 10.37 | 44.9% |

MEDIUM (MODERATE SENSITIVITY)

| | | | | |
|---|---|---|---|---|
| ALL DATA | 297 | 5.8% | 7.35 | 42.3% |
| TYPE 1 | 129† | 8.4% | 4.35 | 36.5% |
| TYPE 2 | 162† | 4.8% | 9.49 | 46.0% |
| BG GROUP 1 | 60 | 11.4% | 5.52 | 63.0% |
| BG GROUP 2 | 160 | 5.6% | 4.92 | 27.4% |
| BG GROUP 3 | 77 | 2.4% | 14.05 | 33.1% |

LOW (LOW SENSITIVITY)

| | | | | |
|---|---|---|---|---|
| ALL DATA | 297 | 3.1% | 9.37 | 28.9% |
| TYPE 1 | 129† | 4.3% | 5.65 | 24.4% |
| TYPE 2 | 162† | 2.7% | 11.96 | 31.7% |
| BG GROUP 1 | 60 | 7.3% | 6.23 | 45.2% |
| BG GROUP 2 | 160 | 2.6% | 6.46 | 16.5% |
| BG GROUP 3 | 77 | 1.3% | 18.50 | 23.2% |

VERY LOW (VERY LOW SENSITIVITY)

| | | | | |
|---|---|---|---|---|
| ALL DATA | 297 | 1.4% | 11.66 | 16.1% |
| TYPE 1 | 129† | 1.6% | 6.11 | 9.8% |
| TYPE 2 | 162† | 1.3% | 15.34 | 19.9% |
| BG GROUP 1 | 60 | 3.5% | 7.54 | 26.6% |
| BG GROUP 2 | 160 | 1.0% | 6.72 | 6.9% |
| BG GROUP 3 | 77 | 0.6% | 26.44 | 15.2% |

Cohorts for glycemic control were BG Group 1: HbA1c<7%, BG Group 2: 7%≦HbA1c<9%, and BG Group 3: HbA1c≧9%. “†” indicates that six (6) Patients were of unknown type and therefore excluded from the diabetes type subgroup analysis.

Method 1's highest flagging frequency predicted approximately 53.5% of BSH is in the Validation dataset. At the lowest flagging frequency setting approximately 16.1% of all BSH in the dataset is predicted, however when a flag is raised there is an approximately 11.66 fold relative probability of a BSH in the next day. In general, across the diabetes type and glycemic control group cohorts shown, Method 1 was predictive of BSH, with the lowest relative probability of an event occurring at the least sensitive setting for Type 1 patients (approximately 3.36 fold likelihood of a BSH event in the next day). Flagging rates for these settings range from approximately 9.7% (approximately once every 10 days on average) to approximately 1.4% (approximately once every two months on average). Approximately 9.7% is slightly lower than the rate for the hypoglycemia risk indicator used in the previously described study, therefore presenting an acceptable upper bound for flagging frequency.

Table 3 below demonstrates the effect of test frequency on performance. We chose the default flagging frequency setting for Method 1 for this analysis. When test frequency ranges are evaluated at this setting, increased BSH sensitivity at higher test frequencies is demonstrated, with a relative probability of detecting a BSH in a day after a flag of more than approximately 14 times for test frequency criteria of ≧approximately 5 times per day.

TABLE 3
The Effect of Test Frequency on Performance

| | Criteria for Average Daily BG Test Frequency in | | | | |
|---|---|---|---|---|---|
| | the Last 14 days Required to Trigger an HRI Flag | | | | |
| | | ≧2.5 and | | | |
| | ≧2 and <2.5 | <3 | ≧3 and <4 | ≧4 and <5 | ≧5 |
| Number of | 17 | 54 | 181 | 159 | 268 |
| BSH | | | | | |
| predicted | | | | | |
| Number of | 246 | 273 | 1568 | 1132 | 1104 |
| calendar | | | | | |
| days with | | | | | |
| at least one | | | | | |
| HRI | | | | | |
| warning | | | | | |
| flag | | | | | |
| Relative | 3.28 | 4.48 | 5.49 | 6.67 | 11.54 |
| probability | | | | | |
| of a BSH in | | | | | |
| the next 24 | | | | | |
| hours after | | | | | |
| a flag | | | | | |

The performance of Method 1 at the medium settings in different test frequency range requirements (I.e. average daily test frequency required over approximately the prior fourteen (14) days in order to trigger an HRI flag). This analysis was performed in the Validation dataset.

These results indicate that the current Method 1 minimum test frequency requirement of approximately 2.5 tests per day (on average) appears to be appropriate. There is satisfactory performance in the approximately 2.5 to approximately 3 tests per day requirement (approximately 54 BSH predicted and a relative probability of BSH of approximately 4.48 in the next 24 hours after a flag), but below this, at test frequencies such as in the approximately 2 to 2.5 per day range, there is poor performance (a relative probability of a BSH of approximately 3.28 in the next 24 hours), and perhaps more importantly there is a paucity of available data (only approximately 17 BSH predicted in this test frequency range). Therefore while larger datasets with more BSH data may allow us to evaluate lower test frequencies with more precision, the average of approximately 2.5 tests per day lower bound requirement appears to be adequate.

As shown in Table 4 below, flagging frequencies near the Method 1 default setting predict significantly more BSH than Methods 2 and 3 (p<approximately 0.05). Method 1 predicted more BSH than Methods 4 and 5 as well, but the results are not statistically significant. It is noteworthy that the performance of Method 2 is well below the other methodologies tested. This finding reinforced previous findings that demonstrated standard deviation's lack of sensitivity to low blood glucose levels, and highlights the need to inform patients and clinicians that standard deviation should only be used for evaluating a propensity for hyperglycemic excursions, or not at all, given the availability of numerous other tools.

TABLE 4
Comparison at flagging frequencies closest to Method 1 default

| | | | Percentage of |
|---|---|---|---|
| | | Relative | all BSH |
| | | Probability of a | occurring |
| | Percentage of | BSH within 24 | within 24 Hours |
| | Days with Flag | Hours of Flag | Following Flag |
| Method 1 | 3.08% | 9.37 | 28.9% |
| Method 2 | 3.02% | 2.24 | 6.8%* |
| Method 3 | 3.11% | 6.18 | 19.2%* |
| Method 4 | 3.10% | 9.06 | 28.1% |
| Method 5 | 3.10% | 8.09 | 25.1%Ψ |

Relative performance of the various methods as measured by the relative probability of BSH within approximately 24 hours of a flag and percentage of all BSH occurring within approximately 24 hours following the flag. “*” indicates there are a lower number of BSH events predicted versus Method 1 and the difference is highly statistically significant (p<approximately 0.001). “Ψ” indicates there are a lower number of BSH events predicted versus Method 1 and the difference is marginally statistically significant (p<approximately 0.10). Statistical significance was evaluated using a chi-square test for proportions.

Table 5 demonstrates the calculated partial area under the receiver operator characteristic for BSH, across a range of useful messaging frequencies. As is demonstrated by the table of calculated areas under the curve in Table 5 below, Method 1 has the highest partial AUROC relative to comparative methods for predicting BSH, thus demonstrating superior performance across the range of messaging frequencies evaluated.

TABLE 5
Partial AUROC calculations for Method 1 and
the four comparative methods

| | Hypoglycemia | BSH |
|---|---|---|
| | (≦70 mg/dL) | (≦40 mg/dL) |
| Method 1 | 0.0260 | 0.0459 |
| Method 2 | 0.0109 | 0.0157 |
| Method 3 | 0.0240 | 0.0394 |
| Method 4 | 0.0244 | 0.0454 |
| Method 5 | 0.0201 | 0.0393 |

AUROC was evaluated for specificities of between 0.875 to 1.0. Method 1 partial AUROC was significantly greater than all other methods evaluated for hypoglycemia (p<approximately 0.01), and for BSH (p<approximately 0.01) Statistical significance was evaluated using the method developed by Hanley and McNeil.

In conclusion, in this study we have evaluated a novel method (Method 1) for warning a patient to near term (24 hour) periods of increased risk of severe hypoglycemia. At a medium setting, Method 1 warned of risk of BSH an average of approximately 5.8% of days in the Validation dataset, predicted approximately 42.3% of BSH within approximately the next 24 hours, and was approximately 7.35 times more likely to be followed by BSH in approximately the next 24 hours than baseline. It was found that Method 1 is more predictive of near term (approximately 24 hour) BSH across a range of flagging frequencies versus the comparative methods used in clinical practice today, or that can be found in the literature.

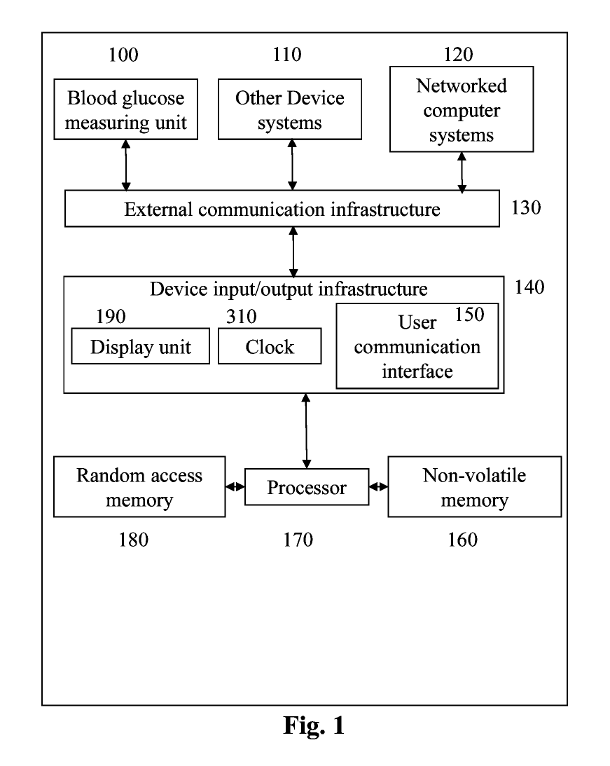

As illustrated, in FIG. 1, certain exemplary embodiments may be implemented in a data management system that interfaces with a blood glucose measuring unit 100 via a unit directly embedded in the data management system or via wire line ports, infrared, fiber optic cable, RF, cellular, Bluetooth, RS 232 or USB cables. An external communication infrastructure 130 accepts blood glucose measurement data from the blood glucose measuring unit 100, and may also have protocols for interfacing with other device systems 110 such as a cellular or hardwire modem, telephone, or fax, and also with networked computer systems 120 such as a local area network, or the Internet also via wire line ports, infrared connections, fiber optic cables, RF, Bluetooth, and/or USB cables. The external communication infrastructure 130 is able to convert information and signals, digital or analog, from external devices and systems into a local machine readable format in the device input/output infrastructure 140, and vice versa.

The device input/output infrastructure 140 can also pre-prepare local machine readable information via encryption or other methods into pre-specified formats for certain users of external systems such as blood glucose measuring units 100, other device systems 110, and/or networked computer systems 120 and also so that it can easily be modified and transmitted by external communication infrastructure 130 or accept encrypted or modified data that it can decipher for processing or saving in non-volatile or random access memory. In addition, device input/output infrastructure 140 preferably has a display unit 190 such as a monitor, speaker, screen or projector for the user to receive information from the system and preferably a user communication interface 150 such as a keyboard, mouse, touchpad, buttons, microphone or camera that allows human control and command of the data management system.

A digital or analog clock 310 with up to date year, day, hour, minute and second information may be accessible through the external communication interface, or imbedded in other system components, but can be located in the device input/output infrastructure 140, and can be remotely adjustable via communication through the external communication infrastructure 130, display unit 190, and/or user communication interface 150.

One or more processors 170 can access instructed program information embedded in the random access memory 180 and non-volatile memory 160 such as a cartridge, memory chip, hard disk drive, flash memory drive, floppy disk drive, magnetic tape drive or optical disk drive, to run programs and route information as instructed, to, from and within device input/output infrastructure 140, as well as access clock 310 information. Processor 170 processes information for output via the display unit 190, through user communication interface 150 and/or through the external communication infrastructure 130.

In an exemplary system, the data management system collects the glucose data from the glucose measuring unit 100, stores it on a computer readable medium such as non-volatile memory 160 or random access memory 180 at the time indicated on the clock 310, uses processor 170 to run a computer program on a computer readable medium that evaluates the newly acquired and stored glucose data, and then if a message is issued according to the program, outputs the message via either display unit 190, user communication interface 150, and/or external communication infrastructure 130.

In an alternative embodiment, at a pre-specified time on clock 310, a program is run. Processor 170 evaluates stored glucose data on the non-volatile memory 160 and determines if a message should be provided to the user on the display unit 190 and/or through external communication infrastructure 130. If the program determines a message should be displayed, the message is displayed at the current time, or if appropriate at a later time contemporaneous with a determined period of risk for hypoglycemia. When the message is received by the user, the user can communicate with processor 170 via user communication interface 150 to learn further details about the message, and contextual information concerning the patterns identified via display unit 190.

In another alternative embodiment, the blood glucose data obtained from a blood glucose measuring unit 100 is saved in a non-volatile memory 160 and processed by processor 170 located external to device input/output infrastructure 140, on other device systems 110, blood glucose measuring unit 100, and/or networked computer systems 120. In such systems when a blood glucose reading is obtained from a blood glucose measuring unit 100, or when the evaluation is otherwise initiated by the system, the blood glucose data or an initiation notification is sent to the external device or system and the evaluation is performed using processor 170, random access memory 180, and non-volatile memory 160 that is beyond the external communication infrastructure 130. Then information is received through the external communication infrastructure 130 from the blood glucose measuring unit, telephonic system or networked computer system performing the evaluation, into the device input/output infrastructure 140 about what information should be messaged to the user, and at which time, and the device input/output infrastructure 140 accesses another internal processor 170 to run a program to proceed to display the message to the user on display unit 190 at the appropriate time as well as save the information in another non-volatile memory 160 internal to the device.

Blood glucose measuring unit 100 can be any device that can function as a blood glucose data acquisition mechanism. The blood glucose measuring or data acquisition mechanism, device, tool or system includes various methods directed towards drawing a blood sample or interstitial fluid sample for each test, and a determination of the glucose level using an instrument that reads glucose concentrations by photometric, electrochemical, or infrared methods. Various proxies for measuring blood glucose can be used such as measuring interstitial fluid glucose or intradermal glucose.

In another alternative embodiment, the device input/output infrastructure 140 accesses another internal processor 170 to run a program to proceed to display all current daily patterns to the user, comprising deleterious glucose patterns and testing deficiency patterns, on display unit 190 at the appropriate time, and the information is saved in another non-volatile memory 160 internal to the device.

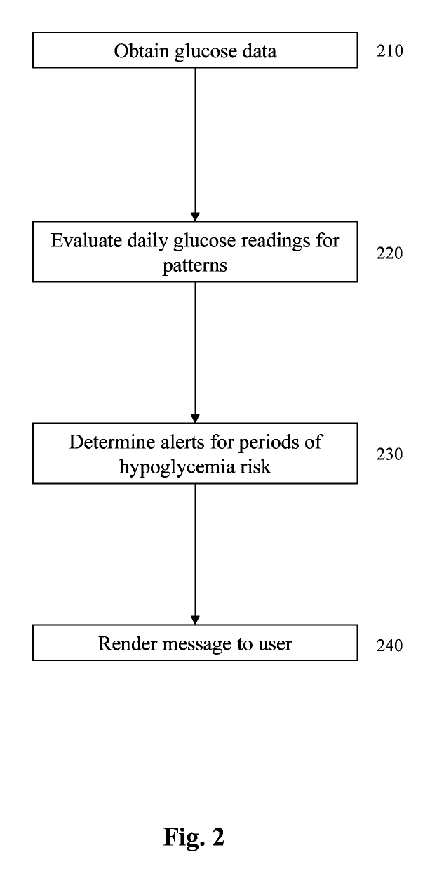

FIG. 2 is a flowchart of an exemplary embodiment of a method adapted to evaluate the patient's risk of severe hypoglycemia, and message the patient in the event that a period has been identified. An exemplary first step 210 is to obtain the glucose data, either from blood glucose measuring unit 100 or networked computer systems 120 (as illustrated in FIG. 1) containing or relaying blood glucose data. An exemplary second step 220 is to evaluate daily glucose readings for patterns, which would involve a processor 170, random access memory 180, and may involve non-volatile memory 160, a blood glucose measuring unit 100, other device systems 110, networked computer systems 120, external communication infrastructure 130, and/or device input/output infrastructure 140 (as illustrated in FIG. 1). In certain exemplary embodiments, method 2000 can comprise automatically determining whether the blood glucose data is sufficient to evaluate a period of hypoglycemia risk.

If a period of risk for hypoglycemia has been identified, an exemplary step 230 is to determine alerts for periods of hypoglycemia risk. In certain exemplary embodiments, a system can be programmed to provide a message or alert in the future about when to test based on analysis of daily time periods wherein risk of hypoglycemia has been identified. More specifically, as a result of a blood glucose reading, a pre-programmed alert, or a user prompt, in a first period of time the system initiates an evaluation of subsequent time periods of the day. Time periods of approximately two (2) hours to approximately forty-eight (48) hours can be evaluated to determine time periods where hypoglycemia risk occurs. Then an exemplary step 240 is to render a message to a user. This message may be rendered contemporaneous with the evaluation of readings and determination of hypoglycemia risk, or at some pre-programmed time in the future.

Certain exemplary methods can comprise via an input/output device, rendering an alert of hypoglycemia risk. The hypoglycemia risk can be determined based upon a strength of an automatically determined pattern in blood glucose data. The blood glucose data can be collected over a time period of at least approximately twenty-four (24) hours. In certain exemplary embodiments, the blood glucose data is collected over a period of between approximately twenty-four (24) hours and four (4) weeks. The automatically determined pattern determined based upon a calculation of at least two metrics selected from:
a first patient metric indicative of a calculated risk value, the calculated risk value determined based upon blood glucose levels deviating towards low blood glucose;
a second patient metric, the second patient metric indicative of a degree of glucose variability; and/or
a third patient metric, the third patient metric indicative of the proportion or relative distribution of glucose readings that are considered hypoglycemic within a given evaluation window.

In certain exemplary embodiments, the patient metric based upon blood glucose levels deviating towards low blood glucose is an LBGI. In certain exemplary embodiments, the patient metric indicative of the degree of glucose variability is an ADRR. In certain exemplary embodiments, a first determination of the strength of the automatically determined pattern can be based on whether at least two of the patient metrics exceed a first set of predetermined thresholds related to each metric used in determining the pattern.

In certain exemplary embodiments, a second determination of the strength of the automatically determined pattern is based on whether at least two of the patient metrics exceed a second set of predetermined thresholds related to each metric used in determining the pattern.

In certain exemplary embodiments, the alert is rendered on a device if at least one of the first or second determinations of the strength of the automatically determined patterns exceeds the predetermined thresholds.

In certain exemplary embodiments, the alert:
advises a diabetic user of a deficiency in the blood glucose data;
advises a diabetic user of an increased risk of hypoglycemia during a predetermined time period;
advises a diabetic user to test blood glucose within a In certain exemplary embodiments, the time period;
advises a diabetic user to have fast acting carbohydrate foods available;
advises a diabetic user to have a glucagon injection kit available;
advises a diabetic user to maintain blood glucose levels above a predetermined threshold;
advises a diabetic user to avoid intense exercise; and/or
advises a diabetic user to eat larger meals or snack more often; and/or comprises tabulated notifications of patterns of hypoglycemic risk.

Certain exemplary methods can comprise, via an input/output device, rendering an alert of hypoglycemia risk. The hypoglycemia risk can be determined based upon a strength of an automatically determined pattern in blood glucose data. The blood glucose data can be collected over a time period of at least 24 hours. The automatically determined pattern determined based upon a calculation of at least two metrics selected from:
a first patient metric indicative of a calculated risk value, the calculated risk value determined based upon blood glucose levels deviating towards low blood glucose;
a second patient metric, the second patient metric indicative of a degree of glucose variability; and/or
a third patient metric, the third patient metric indicative of the proportion or relative distribution of glucose readings that are considered hypoglycemic within a given evaluation window.

In certain exemplary embodiments, a first determination of the strength of the automatically determined pattern is based on whether at least two of the patient metrics exceed a set of predetermined thresholds related to each metric used. In certain exemplary embodiments, the alert can comprise:
a determination that the strength of the automatically determined pattern exceeds a predetermined strength threshold;
a tabulation of historical alerts issued from a device; and/or
blood glucose data preceding and following a time period of at least one prior alert issued from the device.

Certain exemplary methods can comprise, via an input/output device, rendering an alert of hypoglycemia risk. The hypoglycemia risk can be determined based upon a strength of an automatically determined pattern in blood glucose data. The blood glucose data can be collected over a time period of at least 24 hours. The automatically determined pattern can be determined based upon a calculation of at least two metrics selected from:
a first patient metric indicative of a calculated risk value, the calculated risk value determined based upon blood glucose levels deviating towards low blood glucose;
a second patient metric, the second patient metric indicative of a degree of glucose variability; and/or
a third patient metric, the third patient metric indicative of the proportion or relative distribution of glucose readings that are considered hypoglycemic within a given evaluation window.

In certain exemplary embodiments, a user has an option of selecting from two or more hypoglycemia alert settings, and each alert setting changes a set of predetermined thresholds for the at least two metrics. In certain exemplary embodiments, a determination of the strength of the automatically determined pattern is based on whether the at least two metrics exceed thresholds selected from the set of predetermined thresholds related to each metric used. In certain exemplary embodiments, the alert comprises a warning issued from a device indicating identification of the pattern if the determination of the strength of the automatically determined pattern exceeds one or more of the set of predetermined thresholds.

The message could be issued using a device input/output infrastructure 140 that includes a display unit 190 or a user communication interface 150. The message may also be delivered across one or more other device systems 110 or blood glucose measuring units 100 (as illustrated in FIG. 1). Messages conveyed to the user, such as an alert notifying them of a period of risk for hypoglycemia, could be communicated to the user via a display unit 190 that can provide information to the user about patterns and timing, via text, graphical, audible means or other notification means, but preferably via text and graphical means (as illustrated in FIG. 1). In certain exemplary embodiments, the user can access more detailed information about the identified patterns or periods of risk on a separate screen or interface via interaction with a user communication interface 150 such as a touch screen (as illustrated in FIG. 1).

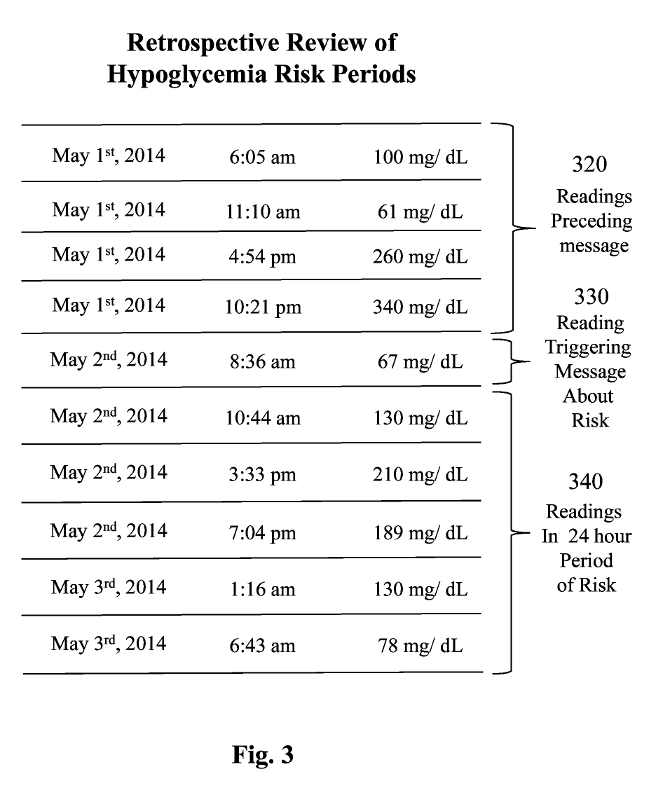

FIG. 3 is an exemplary chart demonstrating the presentation of one or more retrospective messages relating to periods of risk for hypoglycemia. In the chart, readings preceding the period of risk for hypoglycemia 320, or the message indicating the period of risk for hypoglycemia, is provided to the user so they can evaluate their glycemic control leading up to the period of risk. The reading triggering a message about risk 330 such as notification of the entry into the period of risk is rendered so that the user can determine a beginning of the period of risk, and/or when they crossed the threshold into the period of risk. The readings during the period of risk 340 are also shown so that the user can examine glycemic management during the period of risk. In some exemplary embodiments, the readings during the period of risk 340 may extend beyond the risk period, or the risk period may be elongated if the rolling window of data analyses reveals that the period of risk should be extended. The time periods preceding 320, triggering a message about risk 330 and during the period of risk 340 may be highlighted by graphical or other means for easy differentiation by the patient.

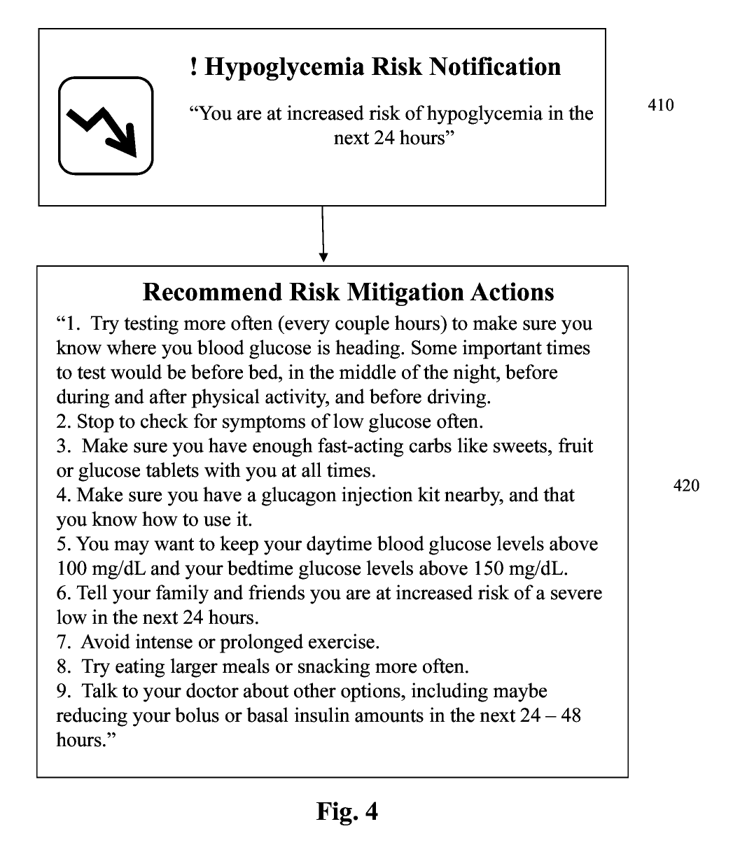

FIG. 4 is an exemplary embodiment of the presentation of a message about a period of risk 410. The presentation preferably includes graphical or iconographic means of presenting a period of risk for hypoglycemia. The presentation may also include text describing the period of risk in detail, comprising precise time ranges or anticipated probability of hypoglycemia. In some embodiments, calculated parameters may also be provided around glucose variability, hypoglycemia frequency, or other metrics. The presentation preferably has a means to access a list of avoidance options for hypoglycemia 420. These may be listed in text or shown in iconographic formats.

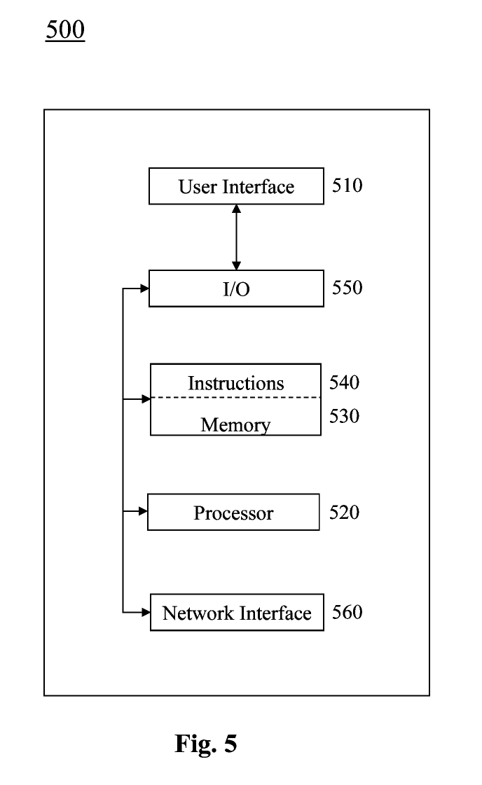

FIG. 5 is a block diagram of an exemplary embodiment of an information device 500. Information device 500 can comprise any of numerous circuits and/or components, such as for example, one or more user interfaces 510, one or more processors 520, one or more memories 530 containing instructions 540, one or more input/output (I/O) devices 550, and/or one or more network interfaces 560 coupled to I/O device 550, etc.

Certain exemplary embodiments may message users about periods of inadequate testing for the evaluation of hypoglycemia risk, identifying where there may not be sufficient information to determine a period of risk for hypoglycemia. Certain exemplary embodiments evaluate and send messages about inadequate testing or periods of risk for hypoglycemia in prospective time periods, so that the user has the opportunity to take action and remedy any deficiency in data.

### DEFINITIONS

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.
a—at least one.
activity—an action, act, step, and/or process or portion thereof.
adapted to—made suitable or fit for a specific use or situation.
ADRR—Average Daily Risk Range, or ADRR, is an index of glucose variability that identifies a daily risk range, or maximum extent of risk-based quantification of low and high blood glucose readings, and takes an average over a pre-defined time period (as further defined in Leon S Farhy, Ph.D., Edward A Ortiz, M.D., Boris P Kovatchev, Ph.D., Alejandra G Mora, B.S., Steven E Wolf, M.D., and Charles E Wade, Ph.D., “Average Daily Risk Range as a Measure of Glycemic Risk Is Associated with Mortality in the Intensive Care Unit: A Retrospective Study in a Burn Intensive Care Unit”, J Diabetes Sci Technol. 2011 September; 5(5): 1087-1098.
advise—to provide information.
alert—a warning.
and/or—either in conjunction with or in alternative to.
apparatus—an appliance or device for a particular purpose
associate—to join, connect together, and/or relate.
automatically—acting or operating in a manner essentially independent of external influence or control. For example, an automatic light switch can turn on upon “seeing” a person in its view, without the person manually operating the light switch.
based upon—determined in consideration of.
blood glucose—a concentration of sugar in human blood.
bolus insulin—the administration of a discrete amount of insulin in order to raise its concentration in blood to an effective level.
calculate—to determine via a mathematical method.
can—is capable of, in at least some embodiments.
collect—to obtain.
comprising—including but not limited to.
configure—to make suitable or fit for a specific use or situation.
convert—to transform, adapt, and/or change.
data—distinct pieces of information, usually formatted in a special or predetermined way and/or organized to express concepts.
deficiency—insufficient or lacking in something.
define—to establish the outline, form, or structure of.
degree—a magnitude of a difference between a determined value and a predetermined threshold.
determine—to obtain, calculate, decide, deduce, and/or ascertain.
deviate—vary.
device—a machine, manufacture, and/or collection thereof.
device systems—a device that outputs information on a user interface or via connected electronic means. Device systems may include a telephone, radio, wearable monitor, biosensor, drug delivery system, data relay, internet hub, PDA, tablet, computer, calculator, messaging system or other.
diabetic user—a person that has a metabolic disease in which the person body is unable to produce any or enough insulin thereby causing elevated levels of glucose in the blood unless medication is used.
estimate—to calculate and/or determine approximately and/or tentatively.
evaluate—to determine or calculate.
evaluation window—a time period during which patient determinations are made.
fast acting carbohydrate food—an edible substance that comprises carbohydrates that are substantially immediately converted to blood sugar when ingested (e.g., candy, soda, orange juice, fruit, raisins, milk, etc.).
following—to go after in time.
frequency—how often something occurs.
generate—to create, produce, give rise to, and/or bring into existence.
glucagon injection kit—a system constructed to intravenously provide to a human a hormone secreted by the pancreas that acts in opposition to insulin in the regulation of blood glucose levels.
haptic—involving the human sense of kinesthetic movement and/or the human sense of touch. Among the many potential haptic experiences are numerous sensations, body-positional differences in sensations, and time-based changes in sensations that are perceived at least partially in non-visual, non-audible, and non-olfactory manners, including the experiences of tactile touch (being touched), active touch, grasping, pressure, friction, traction, slip, stretch, force, torque, impact, puncture, vibration, motion, acceleration, jerk, pulse, orientation, limb position, gravity, texture, gap, recess, viscosity, pain, itch, moisture, temperature, thermal conductivity, and thermal capacity.
HBGI—High Blood Glucose Index, or HBGI, is an index representing the extent and frequency of high blood glucose excursions (as further defined in Kovatchev B P, Cox D J, Gonder-Frederick L A, Clarke W, “Symmetrization of the blood glucose measurement scale and its applications”, Diabetes Care. 1997 November; 20(11):1655-8).
historical—based upon events that occurred in a prior time period that is at least two hours earlier than a referenced time.
HRI—Hypoglycemia Risk Indicator, or HRI, is an alert or warning to increased risk of hypoglycemia within a pre-defined time period.
hypoglycemia—a condition in which a person has a blood sugar level below a normal threshold (e.g., a level of 70 milligrams per deciliter or less).
information device—any device capable of processing data and/or information, such as any general purpose and/or special purpose computer, such as a personal computer, workstation, server, minicomputer, mainframe, supercomputer, computer terminal, laptop, wearable computer, and/or Personal Digital Assistant (“PDA”), mobile terminal, Bluetooth device, communicator, “smart” phone (such as a Treo-like device), messaging service (e.g., Blackberry) receiver, pager, facsimile, cellular telephone, a traditional telephone, telephonic device, a programmed microprocessor or microcontroller and/or peripheral integrated circuit elements, an ASIC or other integrated circuit, a hardware electronic logic circuit such as a discrete element circuit, and/or a programmable logic device such as a PLD, PLA, FPGA, or PAL, or the like, etc. In general any device on which resides a finite state machine capable of implementing at least a portion of a method, structure, and/or or graphical user interface described herein may be used as an information device. An information device can comprise components such as one or more network interfaces, one or more processors, one or more memories containing instructions, and/or one or more input/output (I/O) devices, one or more user interfaces coupled to an I/O device, etc.
input/output (I/O) device—any sensory-oriented input and/or output device, such as an audio, visual, haptic, olfactory, and/or taste-oriented device, including, for example, a monitor, display, projector, overhead display, keyboard, keypad, mouse, trackball, joystick, gamepad, wheel, touchpad, touch panel, pointing device, microphone, speaker, video camera, camera, scanner, printer, haptic device, vibrator, tactile simulator, and/or tactile pad, potentially including a port to which an I/O device can be attached or connected.
LBGI—Low Blood Glucose Index, or LBGI, is an index representing the extent and frequency of low blood glucose excursions (as further defined in Kovatchev B P, Cox D J, Gonder-Frederick L A, Clarke W, “Symmetrization of the blood glucose measurement scale and its applications”, Diabetes Care. 1997 November; 20(11):1655-8).
low—below a predetermined threshold.
machine instructions—directions adapted to cause a machine, such as an information device, to perform one or more particular activities, operations, or functions. The directions, which can sometimes form an entity called a “processor”, “kernel”, “operating system”, “program”, “application”, “utility”, “subroutine”, “script”, “macro”, “file”, “project”, “module”, “library”, “class”, and/or “object”, etc., can be embodied as machine code, source code, object code, compiled code, assembled code, interpretable code, and/or executable code, etc., in hardware, firmware, and/or software.
machine readable medium—a physical structure from which a machine can obtain data and/or information. Examples include a memory, punch cards, etc.
may—is allowed and/or permitted to, in at least some embodiments.
measure—a quantity that is determined from a sensor reading.
memory device—an apparatus capable of storing analog or digital information, such as instructions and/or data. Examples include a non-volatile memory, volatile memory, Random Access Memory, RAM, Read Only Memory, ROM, flash memory, magnetic media, a hard disk, a floppy disk, a magnetic tape, an optical media, an optical disk, a compact disk, a CD, a digital versatile disk, a DVD, and/or a raid array, etc. The memory device can be coupled to a processor and/or can store instructions adapted to be executed by processor, such as according to an embodiment disclosed herein.
method—a process, procedure, and/or collection of related activities for accomplishing something.
metric—a standard for evaluating something.
network—a communicatively coupled plurality of nodes. A network can be and/or utilize any of a wide variety of sub-networks, such as a circuit switched, public-switched, packet switched, data, telephone, telecommunications, video distribution, cable, terrestrial, broadcast, satellite, broadband, corporate, global, national, regional, wide area, backbone, packet-switched TCP/IP, Fast Ethernet, Token Ring, public Internet, private, ATM, multi-domain, and/or multi-zone sub-network, one or more Internet service providers, and/or one or more information devices, such as a switch, router, and/or gateway not directly connected to a local area network, etc.
network interface—any device, system, or subsystem capable of coupling an information device to a network. For example, a network interface can be a telephone, cellular phone, cellular modem, telephone data modem, fax modem, wireless transceiver, ethernet card, cable modem, digital subscriber line interface, bridge, hub, router, or other similar device.
new—most recently determined.
non-volatile memory—a form of memory that persists when the power is off. For example hard drives, floppy drives, USB, SSD etc. . . . are all types of non volatile memory. Read only memory modalities are also non volatile, but are a subset that does not meet most of the following stated uses as they generally cannot be written to.
notifications—an alert, message or other form of communication to an end user using email communication, text messages, in-app messages or display changes, audio prompts, haptic feedback, vibration or other means.
option—a rendered selection
patient metric—a value determined from one or more measurements taken to test a human health parameter.
pattern—a set of values forming a consistent or characteristic arrangement.
PERCLOW—a percentage of blood glucose readings that are below a predetermined threshold during a predetermined time period.
physician—a person practicing medicine.
plurality—the state of being plural and/or more than one.
preceding—to go before in time.
predetermined—established in advance.
pre-existing—determined prior to a most recently measurement.
processor—a device and/or set of machine-readable instructions for performing one or more predetermined tasks. A processor can comprise any one or a combination of hardware, firmware, and/or software. A processor can utilize mechanical, pneumatic, hydraulic, electrical, magnetic, optical, informational, chemical, and/or biological principles, signals, and/or inputs to perform the task(s). In certain embodiments, a processor can act upon information by manipulating, analyzing, modifying, converting, transmitting the information for use by an executable procedure and/or an information device, and/or routing the information to an output device. A processor can function as a central processing unit, local controller, remote controller, parallel controller, and/or distributed controller, etc. Unless stated otherwise, the processor can be a general-purpose device, such as a microcontroller and/or a microprocessor, such the Pentium IV series of microprocessor manufactured by the Intel Corporation of Santa Clara, Calif. In certain embodiments, the processor can be dedicated purpose device, such as an Application Specific Integrated Circuit (ASIC) or a Field Programmable Gate Array (“FPGA”) that has been designed to implement in its hardware and/or firmware at least a part of an embodiment disclosed herein.
project—to calculate, estimate, or predict.
proportion—a comparative relation between values (e.g., a ratio).
provide—to furnish, supply, give, and/or make available.
receive—to get as a signal, take, acquire, and/or obtain.
recommend—to suggest, praise, commend, and/or endorse.
relative distribution—a measure of variability compared to a standard value.
render—to make perceptible to a human, for example as data, commands, text, graphics, audio, video, animation, and/or hyperlinks, etc., such as via any visual, audio, and/or haptic means, such as via a display, monitor, electric paper, ocular implant, cochlear implant, speaker, etc.
repeatedly—again and again; repetitively.
request—to express a desire for and/or ask for.
risk—danger.
risk value—a numerical determination indicative of danger.
set—a related plurality.
setting—a choice that is made for a particular patient that establishes criteria from which a determination is made.
signal—information, such as machine instructions for activities and/or one or more letters, words, characters, symbols, signal flags, visual displays, and/or special sounds, etc. having prearranged meaning, encoded as automatically detectable variations in a physical variable, such as a pneumatic, hydraulic, acoustic, fluidic, mechanical, electrical, magnetic, optical, chemical, and/or biological variable, such as power, energy, pressure, flowrate, viscosity, density, torque, impact, force, frequency, phase, voltage, current, resistance, magnetomotive force, magnetic field intensity, magnetic field flux, magnetic flux density, reluctance, permeability, index of refraction, optical wavelength, polarization, reflectance, transmittance, phase shift, concentration, and/or temperature, etc. Depending on the context, a signal and/or the information encoded therein can be synchronous, asynchronous, hard real-time, soft real-time, non-real time, continuously generated, continuously varying, analog, discretely generated, discretely varying, quantized, digital, broadcast, multicast, unicast, transmitted, conveyed, received, continuously measured, discretely measured, processed, encoded, encrypted, multiplexed, modulated, spread, de-spread, demodulated, detected, de-multiplexed, decrypted, and/or decoded, etc.
store—to place, hold, and/or retain data, typically in a memory.
strength—a magnitude relative to a standard threshold value.
substantially—to a great extent or degree.
sufficient—adequate for making a determination.
system—a collection of mechanisms, devices, machines, articles of manufacture, processes, data, and/or instructions, the collection designed to perform one or more specific functions.
tabulated notifications—systematically arranged data.
tabulation—rendered data that has been systematically arranged.
test—to identify or detect based upon a measurement.
time period—an interval of a fixed duration.
transmit—to send as a signal, provide, furnish, and/or supply.
user interface—any device for rendering information to a user and/or requesting information from the user. A user interface can include textual, graphical, audio, video, animation, virtual reality, robotic control and/or haptic elements. A textual element can be provided, for example, by a printer, monitor, display, projector, etc. A graphical element can be provided, for example, via a monitor, display, projector, and/or visual indication device, such as a light, flag, beacon, etc. An audio element can be provided, for example, via a speaker, microphone, and/or other sound generating and/or receiving device. A video element or animation element can be provided, for example, via a monitor, display, projector, and/or other visual device. A haptic element can be provided, for example, via a very low frequency speaker, vibrator, tactile stimulator, tactile pad, simulator, keyboard, keypad, mouse, trackball, joystick, gamepad, wheel, touchpad, touch panel, pointing device, and/or other haptic device, etc. A user interface can include one or more textual elements such as, for example, one or more letters, number, symbols, etc. A user interface can include one or more graphical elements such as, for example, an image, photograph, drawing, icon, window, title bar, panel, sheet, tab, drawer, matrix, table, form, calendar, outline view, frame, dialog box, static text, text box, list, pick list, pop-up list, pull-down list, menu, tool bar, dock, check box, radio button, hyperlink, browser, button, control, palette, preview panel, color wheel, dial, slider, scroll bar, cursor, status bar, stepper, and/or progress indicator, etc. A textual and/or graphical element can be used for selecting, programming, adjusting, changing, specifying, etc. an appearance, background color, background style, border style, border thickness, foreground color, font, font style, font size, alignment, line spacing, indent, maximum data length, validation, query, cursor type, pointer type, autosizing, position, and/or dimension, etc. A user interface can include one or more audio elements such as, for example, a volume control, pitch control, speed control, voice selector, and/or one or more elements for controlling audio play, speed, pause, fast forward, reverse, etc. A user interface can include one or more video elements such as, for example, elements controlling video play, speed, pause, fast forward, reverse, zoom-in, zoom-out, rotate, and/or tilt, etc. A user interface can include one or more animation elements such as, for example, elements controlling animation play, pause, fast forward, reverse, zoom-in, zoom-out, rotate, tilt, color, intensity, speed, frequency, appearance, etc. A user interface can include one or more haptic elements such as, for example, elements utilizing tactile stimulus, force, pressure, vibration, motion, displacement, temperature, etc.
variability—a quantitative extent to which something changes.
via—by way of and/or utilizing.

### Note

Still other substantially and specifically practical and useful embodiments will become readily apparent to those skilled in this art from reading the above-recited and/or herein-included detailed description and/or drawings of certain exemplary embodiments. It should be understood that numerous variations, modifications, and additional embodiments are possible, and accordingly, all such variations, modifications, and embodiments are to be regarded as being within the scope of this application.

Thus, regardless of the content of any portion (e.g., title, field, background, summary, description, abstract, drawing figure, etc.) of this application, unless clearly specified to the contrary, such as via explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
no characteristic, function, activity, or element is “essential”;
any elements can be integrated, segregated, and/or duplicated;
any activity can be repeated, any activity can be performed by multiple entities, and/or any activity can be performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any claim element is followed by a drawing element number, that drawing element number is exemplary and non-limiting on claim scope. No claim of this application is intended to invoke paragraph six of 35 USC 112 unless the precise phrase “means for” is followed by a gerund.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

What is claimed is:

1. A method comprising:
via an input/output device, rendering an alert of hypoglycemia risk, said hypoglycemia risk determined based upon a strength of an automatically determined pattern in blood glucose data, said blood glucose data collected over a time period of at least 24 hours, said automatically determined pattern determined based upon a calculation of at least two metrics selected from:
a first patient metric indicative of a calculated risk value, said calculated risk value determined based upon blood glucose levels deviating towards low blood glucose;
a second patient metric, said second patient metric indicative of a degree of glucose variability; and
a third patient metric, said third patient metric indicative of the proportion or relative distribution of glucose readings that are considered hypoglycemic within a given evaluation window;
wherein a first determination of said strength of said automatically determined pattern is based on whether at least two of said patient metrics exceed a first set of predetermined thresholds related to each metric used in determining said pattern;
wherein a second determination of said strength of said automatically determined pattern is based on whether at least two of said patient metrics exceed a second set of predetermined thresholds related to each metric used in determining said pattern;
wherein said alert is rendered on a device if at least one of said first or second determinations of the strength of said automatically determined patterns exceeds the predetermined thresholds.

2. The method of claim 1, further comprising:
automatically determining whether said blood glucose data is sufficient to evaluate a period of hypoglycemia risk.

3. The method of claim 2, wherein:
said alert advises a diabetic user of a deficiency in said blood glucose data.

4. The method of claim 1, wherein:
said patient metric based upon blood glucose levels deviating towards low blood glucose is an LBGI.

5. The method of claim 1, wherein:
said patient metric indicative of said degree of glucose variability is an ADRR.

6. The method of claim 1, wherein:
said blood glucose data collected over a period of between 24 hours and four weeks.

7. The method of claim 1, wherein:
said alert advises a diabetic user of an increased risk of hypoglycemia during a predetermined time period.

8. The method of claim 1, wherein:
said alert advises a diabetic user to test blood glucose within a predetermined time period.

9. The method of claim 1, wherein:
said alert advises a diabetic user to have fast acting carbohydrate foods available.

10. The method of claim 1, wherein:
said alert advises a diabetic user to have a glucagon injection kit available.

11. The method of claim 1, wherein:
said alert advises a diabetic user to maintain blood glucose levels above a predetermined threshold.

12. The method of claim 1, wherein:
said alert advises a diabetic user to avoid intense exercise.

13. The method of claim 1, wherein:
said alert advises a diabetic user to eat larger meals or snack more often.

14. The method of claim 1, wherein:
said alert comprises tabulated notifications of patterns of hypoglycemic risk.

15. A method comprising:
via an input/output device, rendering an alert of hypoglycemia risk, said hypoglycemia risk determined based upon a strength of an automatically determined pattern in blood glucose data, said blood glucose data collected over a time period of at least 24 hours, said automatically determined pattern determined based upon a calculation of at least two metrics selected from:
a first patient metric indicative of a calculated risk value, said calculated risk value determined based upon blood glucose levels deviating towards low blood glucose;
a second patient metric, said second patient metric indicative of a degree of glucose variability; and
a third patient metric, said third patient metric indicative of the proportion or relative distribution of glucose readings that are considered hypoglycemic within a given evaluation window; and
wherein a first determination of the strength of said automatically determined pattern is based on whether at least two of said patient metrics exceed a set of predetermined thresholds related to each metric used;
wherein said alert comprises:
a determination that said strength of said automatically determined pattern exceeds a predetermined strength threshold;
a tabulation of historical alerts issued from a device; and
blood glucose data preceding and following a time period of at least one prior alert issued from said device.

16. A method comprising:
via an input/output device, rendering an alert of hypoglycemia risk, said hypoglycemia risk determined based upon a strength of an automatically determined pattern in blood glucose data, said blood glucose data collected over a time period of at least 24 hours, said automatically determined pattern determined based upon a calculation of at least two metrics selected from:
a first patient metric indicative of a calculated risk value, said calculated risk value determined based upon blood glucose levels deviating towards low blood glucose;
a second patient metric, said second patient metric indicative of a degree of glucose variability; and
a third patient metric, said third patient metric indicative of the proportion or relative distribution of glucose readings that are considered hypoglycemic within a given evaluation window; and
wherein a user has an option of selecting from two or more hypoglycemia alert settings, and each alert setting changes a set of predetermined thresholds for said at least two metrics;
wherein a determination of said strength of said automatically determined pattern is based on whether said at least two metrics exceed thresholds selected from said set of predetermined thresholds related to each metric used;
wherein said alert comprises a warning issued from a device indicating identification of said pattern if said determination of said strength of said automatically determined pattern exceeds one or more of said set of predetermined thresholds.
